(19) United States
(12) Patent Application Publication
(10) Pub. No.: US 2023/0021923 A1
(43) Pub. Date: Jan. 26, 2023

(54) OPTICAL SWELL METERS AND METHODS OF USING SAME

(71) Applicant: University of Louisiana at Lafayette, Lafayette, LA (US)

(72) Inventors: Mehdi Mokhtari, Lafayette, LA (US); Prathmesh Parrikar, Lafayette, LA (US)

(73) Assignee: University of Louisiana at Lafayette, Lafayette, LA (US)

(21) Appl. No.: 17/867,087

(22) Filed: Jul. 18, 2022

Related U.S. Application Data
(60) Provisional application No. 63/223,331, filed on Jul. 19, 2021.

(51) Int. Cl. G01N 33/24; C09K 8/03

(52) U.S. Cl. CPC G01N 33/24; C09K 8/03; C09K 2208/12

(57) ABSTRACT

Disclosed herein are optical swell meters and methods for using them to measure fine-scale variations in the swelling behavior of shale specimens. In an embodiment, the optical swell meters can include: a glass imbibition chamber containing a specimen, where an imbibition fluid is located within the glass inhibition chamber and at least partially covering the specimen; a digital camera set a distance away from the chamber and facing the chamber; and a light source, where the light source illuminates the specimen. The method of using the optical swell meter can provide a swelling strain profile of the shale specimens during its interaction with the imbibition fluid.

### CROSS REFERENCE TO RELATED APPLICATIONS

This application claims the benefit of priority under 35 U.S.C. § 119(e) of U.S. Ser. No. 63/223,331, filed Jul. 19, 2021, the entire contents of which is incorporated herein by reference in its entirety.

### BACKGROUND

### Field

Disclosed herein are optical swell meters and methods for creating a swelling strain profiles of shale specimens. The optical swell meters can measure fine-scale variations and capture the role of small-scale heterogeneity in the swelling behavior of the shale specimens.

### Description of the Related Art

Interaction of clay-rich formations with fluids occurs during drilling, hydraulic fracturing, and enhanced oil recovery operations. During these operations, clay swelling can cause conductivity damage by impeding hydrocarbon flow. The degree to which damage induced by swelling of the clays prevails, is dependent upon the mineralogy of the rock, composition of fracturing and reservoir fluids, and fluid access to different zones of the fracture network. In the case of wellbore drilling operations for petroleum exploration or extraction, water-based drilling fluids are often used instead of oil- or synthetic-based alternatives due to their environmental effects. Water, however, can react with clays and lead to their hydration and subsequent swelling. This, in turn, may have a negative impact on well costs, and can impede the drilling process.

To minimize the adverse effects of clay swelling during these operations, it is crucial to monitor and understand the mechanisms behind the process. Past research has conducted measurements of swelling in shale when immersed in water. These studies used conventional measurement techniques like linear variable differential transformer (LVDT) sensors and strain gauges, which provided average strain values, but did not investigate fine-scale variation in the deformation of the shale specimens.

Consequently, there is need for new systems and methods for measuring fine-scale variations of shale, capturing the role of small-scale heterogeneity in their swelling.

### SUMMARY

Provided herein are optical swell meters and methods for using them to provide a swelling strain profile of samples during interaction with a fluid. In a specific embodiment, the system can include a glass imbibition chamber containing a specimen, where an imbibition fluid is located within the glass inhibition chamber and at least partially covering the specimen; a digital camera set a distance away from the chamber and facing chamber; and at least one light source, where the light source illuminates the specimen.

In another specific embodiment, a method for creating a swelling strain profile can include: applying a random intensity speckle pattern to at least one face of a specimen, the specimen being capable of deformation due to contact with a fluid; placing a specimen within a glass chamber and filling the glass chamber with an imbibition fluid; illuminating the specimen; capturing a plurality of images of the specimen over time using a digital camera and thereby creating digital images; and using digital image correlation to analyze the digital images to generate a strain map across the specimen surface.

### BRIEF DESCRIPTION OF THE DRAWINGS

The present disclosure can be better understood by referring to the following drawings. The drawings constitute a part of this specification and include exemplary embodiments of the optical swell meters, which can be embodied in various forms.

FIGS. 1A and 1B are a schematic and three-dimensional rendering, respectively, of digital image correlation setup to capture specimen deformation during imbibition.

FIGS. 2A-2B shows average strain and standard deviation of strain observed during underwater DIC calibration (a) in plane displacement (b) out of plane displacement.

FIGS. 3A-3E shows vertical normal strain evolution in S2 specimen with time (a) 0.045 hrs (b) 0.096 hrs (c) 0.367 hrs (d) 0.612 hrs (e) 4.21 hrs.

FIGS. 4A-4B shows horizontal normal strain in S2 at (a) 0.096 hrs (b) 0.367 hrs.

FIG. 5 shows a comparison of vertical normal strains.

FIG. 6 shows displacements in S2 specimens at 100 hrs and showing sections selected for average strain calculations (a) Vertical displacement (b) Horizontal displacement.

FIG. 7 is a graph of average vertical normal strains in the specimens as a function of time.

### DETAILED DESCRIPTION

The optical swell meters can include, but are not limited to: one or more glass chambers, one or more digital camera, and one or more light emitting diode lights. The method of using the optical swell meters can include providing a swelling strain profile of shale samples during interaction with a fluid. The shale specimen to be tested can be painted to generate a random speckle pattern. Then, the shale specimen can be placed in a glass chamber containing a fluid of interest. The digital camera can be used to capture the deformation process as the submerged sample swells. The digital images can be analyzed using a digital image correlation technique to generate strain maps across the specimen surface for every image captured during the swelling process. The strain map can include a collective of strains measurements at thousands of points on the sample surface and, therefore, can provide valuable information about strain distributions and strain localization. The crack formation and propagation during the swelling can also be observed from the strain maps.

The method of using the optical swell meter can include applying a random intensity speckle pattern to the front face (the face towards the camera) of the specimen by using flat white spray paint. The specimen can be placed on a brass wire mesh, which can be attached to the base of the imbibition chamber thus allowing an all-face-open imbibition configuration. The speckle pattern on the specimen can be illuminated using two LED lights. The digital camera can be used to capture images of the swelling of the specimen over time. The captured images can be analyzed using digital image correlation. The digital image correlation enables the visualization and quantification of full-field deformation. Full-field monitoring helps measure the strain distribution and localization, as well as the evolution of strain field with time. The strain localization is influenced by the distribution of the minerals in the specimen. The results provide a better understanding of strain development during imbibition in comparison to traditional linear variable differential transformer-based measurements that give only an average strain value. The results from the study show large strains get localized along a few select laminations. These laminations showed a large tensile strain in the direction perpendicular to the bedding plane.

The optical swell meters and methods for using them allow for measuring fine-scale variations thereby capturing the role of small-scale heterogeneity in the swelling behavior of shale specimens. The optical swell meters provide a means for investigating the swelling deformation of shale upon immersion in water using digital image correlation. The digital image correlation is a non-contact measurement technique developed for capturing deformations over the surface as a function of time.

The optical swell meters can measure the deformation of clay-rich geologic rocks in contact with water using camera associated with digital image correlation techniques. The optical swell meters can capture the full-field deformation of shale compared to the average values provided with the linear variable differential transformer methods, thus the optical swell meter can be more representative of heterogeneous nature of shale formation (democratization of deformation) with shales being anisotropic in nature (behave differently in different directions). The optical swell meters can provide swelling measurements in the direction of lamination versus perpendicular to lamination. To represent the complex nature of shale formation for improved engineering designs, the optical swell meters can provide the full-field deformation of shale due to water interaction. The optical swell meters can support geothermal, hydrocarbon and carbon dioxide companies to mitigate wellbore collapse, carbon dioxide leakage, and more informed decisions on field development.

In an embodiment, the method of using the optical swell meters can include a digital image correlation technique as a non-contact method for full-field measurement of microscopic shale deformation in interaction with water. A speckle pattern is applied to the specimen and then the deformation of the specimen during imbibition is captured using a digital camera. The images are analyzed using a 2D-digital image correlation image processing software to obtain displacements and strains. The method of using the optical swell meters can yield measurements of deformation that evolve over time, showing the role of each lamina on deformation.

### EXAMPLES

To provide a better understanding of the foregoing discussion, the following non-limiting examples are offered. Although the examples can be directed to specific embodiments, they are not to be viewed as limiting the invention in any specific respect. Examples of the graft copolymers of dextran and polyacrylamide and reaction conditions for making them are shown in Tables 1-6.

### Example 1

Tuscaloosa Marine Shale samples with 35% to 52% clay are tested in interaction with water. Implementation of digital image correlation technique to capture deformation during the imbibition process and the impact of clay content on fine-scale deformation and consequent fracturing of clay-rich shales.

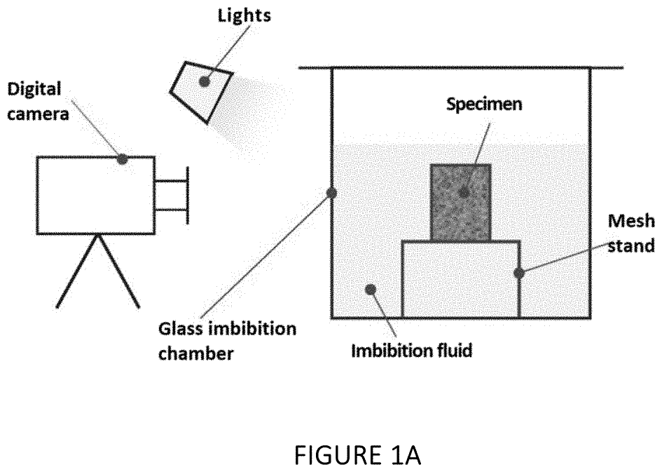

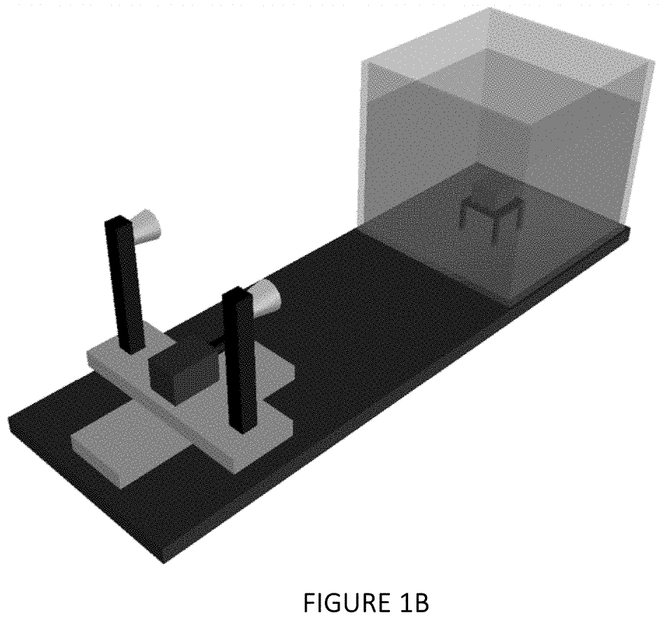

The digital image correlation can capture the specimen deformation during imbibition as shown in FIG. 1. The specimen sits on a brass wire mesh, which is attached to the base of the imbibition chamber thus allowing an all-face-open imbibition configuration. The specimen is placed on the brass mesh and then deionized water is introduced in the chamber till the specimen is submerged.

A 6.3 MP monochrome digital camera (Blackfly S) was used to capture the deformation images while the specimen in submerged in DI water. The images were taken at 5-minute interval for the first 2 hrs and then 30-minute interval for 4 days. The images were analyzed using commercial DIC software (ARAMIS 2019). The DIC analysis was conducted using a subset size of 45 pixels and a step size of 15 pixels.

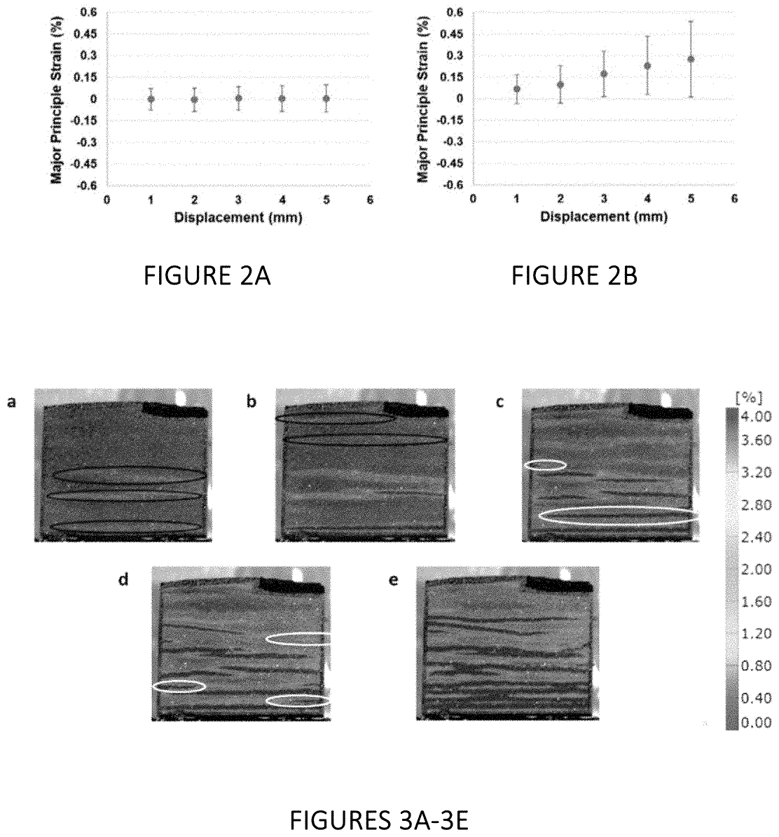

A calibration study was conducted to evaluate the accuracy of the underwater 2D-DIC system. A non-deforming flat aluminum target plate with a random speckle pattern was placed underwater in the location of the specimen for the experiment. The aluminum target was given precise in-plane (in the image plane—perpendicular to the optical path) and out-of-plane (along optical path) displacements using a micrometer. Images of the aluminum target plate were captured at different displacements and then a deformation analysis was conducted using DIC to quantify the effect of prescribed translation on pseudo-DIC stain. The strains observed in the calibration study are shown in FIG. 2. The observed average principle strain and standard deviations during in-plane displacement were not sensitive to displacement input and their value was below 0.1%. Out-of-plane displacement caused larger pseudo strains and the strains increased with increasing displacements. To minimize the errors in strain measurements in the swelling experiments the highest swelling direction (perpendicular to bedding planes) was aligned with in-plane direction (vertical direction).

The two specimens for this example were selected from different depths of a single well in TMS formation. X-ray diffraction analysis was conducted on selected samples to identify mineralogy. The results are presented in Table 1.

TABLE 1
X-Ray Diffraction Analysis

| Name | Depth, ft | Quartz | K-Feldspars | Plagioclase | Calcite | Pyrite | Siderite | Total Clay |
|---|---|---|---|---|---|---|---|---|
| S1 | 12182 | 58.2 | 0.4 | 3.7 | 0.6 | 2.4 | 0.3 | 34.4 |
| S2 | 12142 | 32.7 | 0 | 2.1 | 8.8 | 4.6 | 0.5 | 51.3 |

Both the selected specimens were rich in clay, with a weight percentage of 34.4 in specimen S1 and 52.2 in S2. Quartz was the other major constituent with S1 having 58.2 and S2 having 37.7 wt %.

For this example, shale samples were cut into cuboid shape with bedding planes oriented horizontally, and nominal specimen size of 38 mm width, 25 mm height and 25 mm depth. The DIC system allowed the monitoring of deformation over the entire speckled surface of the specimen and evaluation of the deformation as a function of location and time.

Both the specimens exhibited large swelling in the vertical direction. FIG. 3 shows the normal strain in the vertical direction (ε_{yy}), in specimen S2 and how it evolves with time. The images displayed were selected to showcase the deformation behavior and have varying timestamps. The time of introduction of imbibition fluid was taken as time 0. The deformation evolution was tracked with respect to this time 0.

FIG. 3a shows ε_{yy} in S2 at 0.045 hrs. Black bubbles mark the sites in S2 that were first to undergo large swelling after interaction with water and display a strain magnitude of more than 2%. These swelling sites have a horizontal shape. After 0.096 hrs (FIG. 3b) the sites of initial swelling were seen to have grown in size and magnitude with some of the sites of initial swelling reaching magnitudes above 4%. Also, some more initial swelling sites (marked in black bubbles) were observed to develop in the top portion of the specimen.

FIG. 3c shows ε_{yy} observed at 0.367 hrs where new swelling sites (highlighted in white bubbles) were seen in the close vicinity of initial swelling sites and had already swollen to a large magnitude. Some of these locations of large strain are cracks that were interpreted as large strain in DIC software. The new sites undergoing swelling also reach high strain magnitudes (magnitude greater than 4%) and after 0.612 hrs were seen to stimulate another set of new sites of swelling in their neighborhood (FIG. 3d marked with white bubbles). These new secondary sites were again seen to develop in the vicinity of existing high swollen sites and had horizontal laminar shape. FIG. 3e shows ε_{yy} in S2 after 4.21 hrs. The initial and stimulated secondary swelling sites had grown and display large strains over 4%. Strain values of about 2% were observed in the region between the high swelling sites across the entire surface.

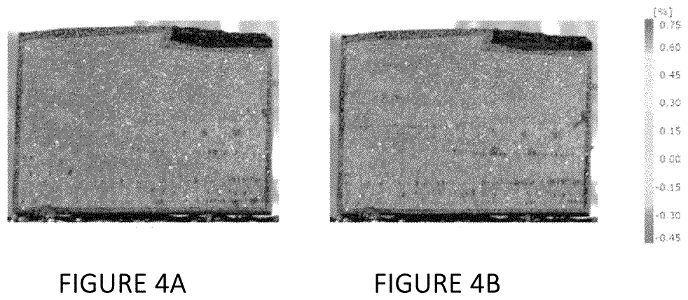

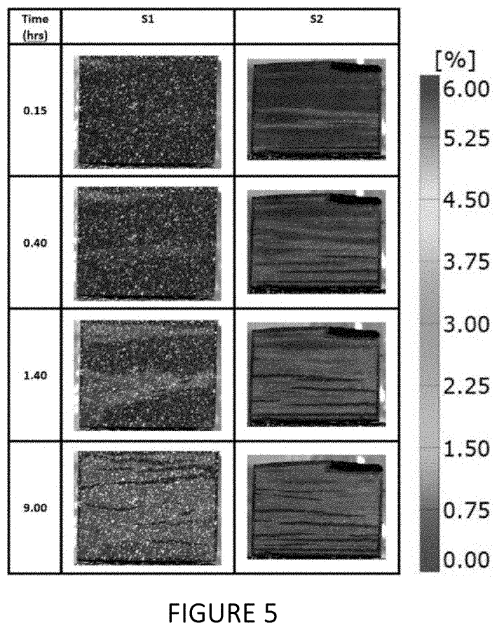

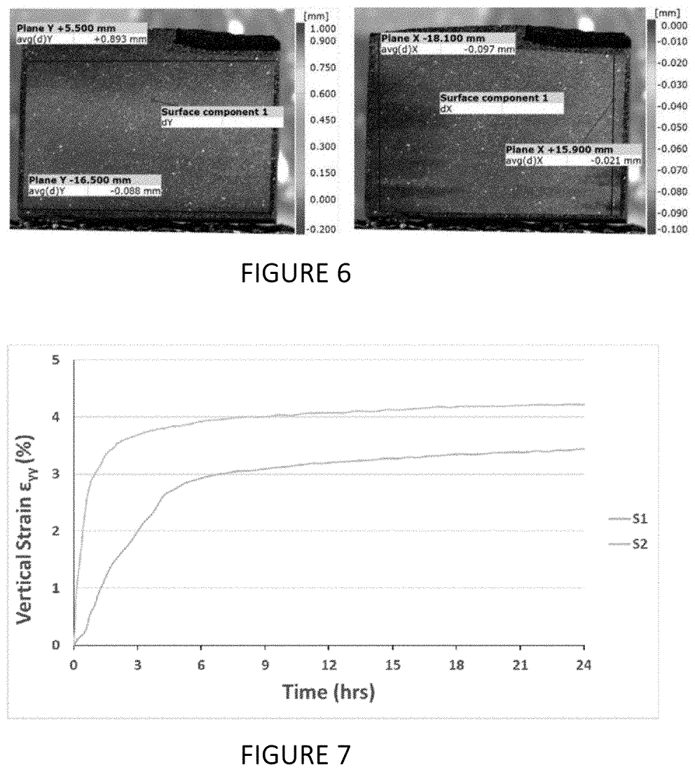

Horizontal normal strains (εₓₓ) in S2 at 0.096 hrs and 0.367 hrs are shown in FIG. 4. The εₓₓ values observed are much lower than corresponding C_{yy} values in FIG. 3b and FIG. 3c. Some of the sites with high ε_{yy} values in FIG. 3 showed positive and negative εₓₓ strains. These regions are mostly sites that got cracked and the changes in speckle pattern due to the introduction of crack resulted in alternating positive and negative pseudo cxx strains patterns.

FIG. 5 shows vertical normal strains measured on the face of both the specimens at four different times. At 0.15 hr strains in S1 were low and uniform across the specimen while S2 already showed some initial swelling sites. Some of the initial swelling sites in S2 showed high strains of more than 6% after 0.15 hrs. At 0.40 hrs, two sites of initial swelling were seen in S1 whereas, in S2 multiple initial swelling sites and some secondary swelling sites were observed. Most of the initial swelling sites observed in S2 at 0.40 hrs showed high strains with magnitudes greater than 6%. At 1.40 hrs the initial swelling sites in S1 were seen to reach higher strains. S2 showed initial and secondary swelling sites reaching high strains of more than 6% after 1.40 hrs. The region between high swelling sites in S2 also showed significant swelling, swelling of 2-3%, at 1.40 hrs. At 9.00 hrs, both specimens showed primary and secondary swelling sites with high strains of more than 6%. Specimen S2 showed a large number of high swelling sites in comparison to S1. The regions between high swelling sites in S2 showed significantly larger swelling in comparison to S1.

Average normal vertical strain and average normal horizontal strain were calculated for the specimens to compare their swelling behavior as a function of time. The average strain in each specimen was calculated by tracking the average displacement of sections at specimen boundaries. FIG. 5 shows the displacement in the vertical and horizontal direction in the S2 specimen after 100 hrs along with the sections used to measure average stains. Horizontal sections were selected near the top and bottom boundary of the specimen and the average vertical displacements of the sections were used to measure vertical strain. Similarly, vertical sections were selected near the left and right boundary of the specimen and the average horizontal displacement of the vertical sections was used to measure average horizontal strain in the specimen. The average strain was calculated using the following equation (1): ε=Δl/l where Δl is the difference in average displacements of the two vertical sections or two horizontal sections and l is the original distance between the two sections at time 0.

The evolution of average vertical strains in the two specimens is shown in FIG. 7. Specimen S2 showed a very high increase in vertical strains during the first 0.5 hrs in comparison to S1. This is because S2 had multiple initial and secondary swelling sites reach high strain while the initial swelling sites in S1 were still in the incipient stage. The high swelling sites in S1 develop slowly resulting in a slow increase of average strain in the specimen. The rate of swelling and magnitude of vertical strain during the beginning of deformation appears to be influenced strongly by the clay content of the specimen. The specimen with high clay content showed higher strain. The average vertical strain increased rapidly in the initial 6 hrs and followed by a slow increase in strain. The majority of the high swelling sites developed during this initial 6 hrs. The further increase in strains in specimens after 6 hrs is associated with swelling of regions between high swelling sites in the specimens.

The results show that the swelling deformation in specimens, when exposed to deionized water, is not uniform. Large numbers of high swelling sites were observed in the specimens (FIG. 3 and FIG. 5). These sites with high ε_{yy} had horizontal laminar shape and are most likely lamina layers with high clay content. As the deformation grew larger, some of these high swelling sites developed cracks. The cracks were in the middle of the high swelling sites. The deformation of all the high swelling sites occurred at different time frames. The initial high swelling sites showed swelling early in the deformation while the secondary high swelling sites started swelling only after being stimulated by another high swelling site in their neighborhood. The magnitudes of swelling were similar in initial and secondary swelling sites. The rate of swelling in initial and secondary sites in any given specimen after the triggering of swelling was also similar. This indicates that both initial and secondary swelling sites have high clay content and have similar swelling behavior. The different times of activation of swelling in individual swelling sites must be dependent on the fabric of the specimen and the presence of crack surfaces. The initial swelling sites might have preexisting cracks that trigger the swelling deformation in them upon exposure to water. The deformation in initial swelling sites along with the cracking in the initial swelling sites caused stimulation of secondary swelling sites that had high clay content.

To understanding the optical swell meter, references are made in the text to exemplary embodiments of an optical swell meter, only some of which are described herein. It should be understood that no limitations on the scope of the invention are intended by describing these exemplary embodiments. One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

Although the invention has been described with reference to the above examples, it will be understood that modifications and variations are encompassed within the spirit and scope of the invention. Accordingly, the invention is limited only by the following claims.

What is claimed is:

1. An optical swelling meter comprising:
a glass imbibition chamber containing a specimen, wherein an imbibition fluid is located within the glass inhibition chamber and at least partially covering the specimen;
a digital camera set a distance away from the chamber and facing the chamber; and
an at least one light source, wherein the light source illuminates the specimen.

2. The system of claim 1 wherein the imbibition fluid is deionized water.

3. The system of claim 1 wherein the specimen rests on a brass wire mesh that is attached to the base of the glass imbibition chamber.

4. A method for creating a swelling strain profile comprising:
applying a random intensity speckle pattern to at least one face of a specimen, the specimen being capable of deformation due to contact with a fluid;
placing a specimen within a glass chamber and filling the glass chamber with an imbibition fluid;
illuminating the specimen;
capturing a plurality of images of the specimen over time using a digital camera and thereby creating digital images; and
using digital image correlation to analyze the digital images to generate a strain map across the specimen surface.
